(19) United States
(12) Patent Application Publication
(10) Pub. No.: US 2010/0137842 A1
(43) Pub. Date: Jun. 3, 2010

(54) Ambulatory Infusion Devices With Improved Delivery Accuracy

(76) Inventor: Scott R. Gibson, Granada Hills, CA (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 12/326,336

(22) Filed: Dec. 2, 2008

(51) Int. Cl. A61K 9/22

(52) U.S. Cl. 604/890.1

(57) ABSTRACT

Infusion devices that are configured to measure fluid flow and to adjust delivery.

### BACKGROUND

1. Field

The present devices and methods relate generally to ambulatory infusion devices such as, for example, implantable infusion devices.

2. Description of the Related Art

Ambulatory infusion devices have been used to provide a patient with a medication or other substance (collectively “infusible substance”) and frequently include a reservoir and a pump. The reservoir is used to store the infusible substance and, in some instances of implantable infusion devices, a fill port is provided that allows the reservoir to be transcutaneously filled (and/or re-filled) with a hypodermic needle. The reservoir is coupled to the pump, which is in turn connected to an outlet port. A catheter or other device, which has at least one outlet at the target body region, may be connected to the outlet port. As such, infusible substance may be transferred from the reservoir to the target body region by way of the pump and catheter.

Delivery accuracy, i.e. the delivery of the intended volume of infusible substance to the patient, is an important aspect of any infusion device. The present inventor has determined that a number of factors may adversely effect delivery accuracy. The accuracy of the pump may, for example, degrade over time. Variations in temperature, reservoir pressure, and pressure at the outlet port may also adversely effect delivery accuracy. Degradation of catheter patency is another factor that can adversely effect delivery accuracy.

### SUMMARY

At least some of the present infusion devices and methods determine the actual volumetric flow from fluid transfer device actuations and adjust the number of subsequent actuations in response to the actual volumetric flow being different than the expected volumetric flow. There are a variety of advantages associated with such devices and methods. By way of example, but not limitation, the present devices and methods compensate for factors adversely effecting delivery accuracy, in what is essentially real time, on an ongoing basis.

At least some of the present infusion devices and methods calculate volumetric flow by determining the pressure differential across a flow restrictor. Pressure on one side of the flow restrictor may be measured directly, while pressure on the other side may be measured indirectly, thereby eliminating the expense that would have been associated with a second pressure sensor.

The above described and many other features of the present devices and methods will become apparent as the devices and methods become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a plan view of an implantable infusion device in accordance with one embodiment of a present invention.

FIG. 2 is a plan view of the exemplary implantable infusion device illustrated in FIG. 1 with the cover removed.

FIG. 3 is a partial section view taken along line 3-3 in FIG. 1.

FIG. 4 is a block diagram of the exemplary implantable infusion device illustrated in FIGS. 1-3.

FIG. 5 is a section view of a flow restrictor in accordance with one embodiment of a present invention.

FIG. 6 a graph showing a current waveform associated with the actuation of a fluid transfer device.

FIGS. 7 and 8 are graphs showing exemplary delivery profiles.

FIG. 9 is a flow chart in accordance with one embodiment of a present invention.

FIG. 10 is a plan view of an implantable infusion device system in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also not limited to the exemplary implantable infusion devices described herein and, instead, are applicable to other implantable or otherwise ambulatory infusion devices that currently exist or are yet to be developed.

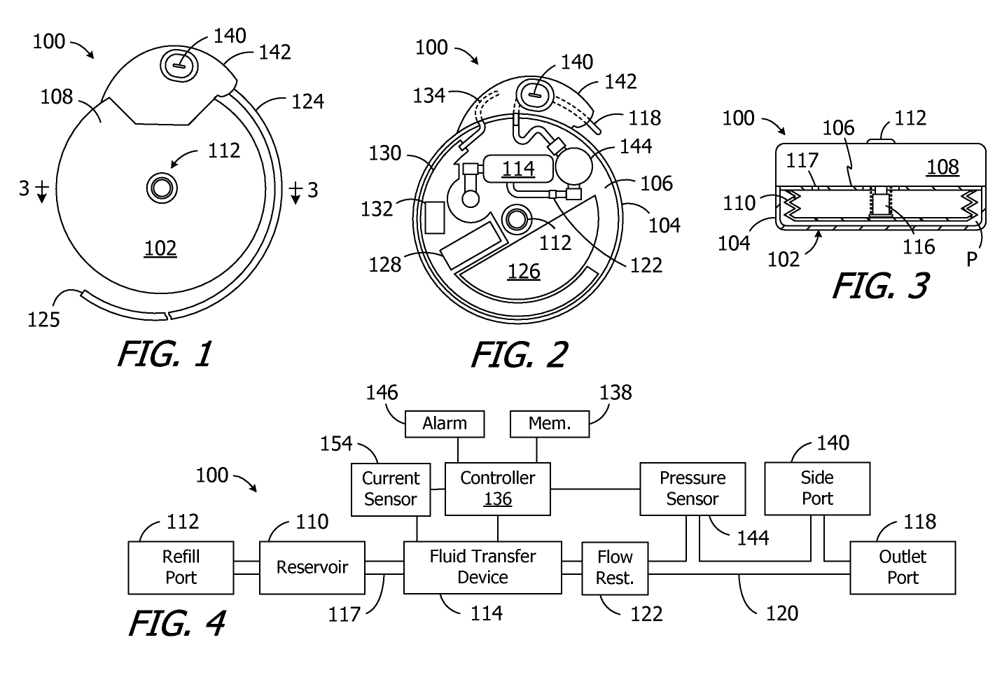

One example of an implantable infusion device in accordance with a present invention is generally represented by reference numeral 100 in FIGS. 1-4. As used herein, an “implantable infusion device” is a device that includes a reservoir and an outlet, and is sized, shaped and otherwise constructed (e.g. sealed) such that both the reservoir and outlet can be simultaneously carried within the patient's body. The exemplary infusion device 100 includes a housing 102 (e.g. a titanium housing) with a bottom portion 104, an internal wall 106, and a cover 108. An infusible substance (e.g. medication) may be stored in a reservoir 110 that is located within the housing bottom portion 104. The reservoir 110 may be replenished by way of a fill port 112 that extends from the reservoir to the cover 108. A hypodermic needle (not shown), which is configured to be pushed through the fill port 112, may be used to replenish the reservoir 110. Fluid flow from the fill port 112 to the reservoir 110 may be controlled by an inlet valve (not shown).

A wide variety of reservoirs may be employed. In the illustrated embodiment, the reservoir 110 is in the form of a titanium bellows that is positioned within a sealed volume defined by the housing bottom portion 104 and internal wall 106. The remainder of the sealed volume is occupied by propellant P, which may be used to exert negative pressure on the reservoir 110. Other reservoirs that may be employed in the present infusion devices include reservoirs in which propellant exerts a positive pressure. Still other exemplary reservoirs include negative pressure reservoirs that employ a movable wall that is exposed to ambient pressure and is configured to exert a force that produces an interior pressure that is always negative with respect to the ambient pressure.

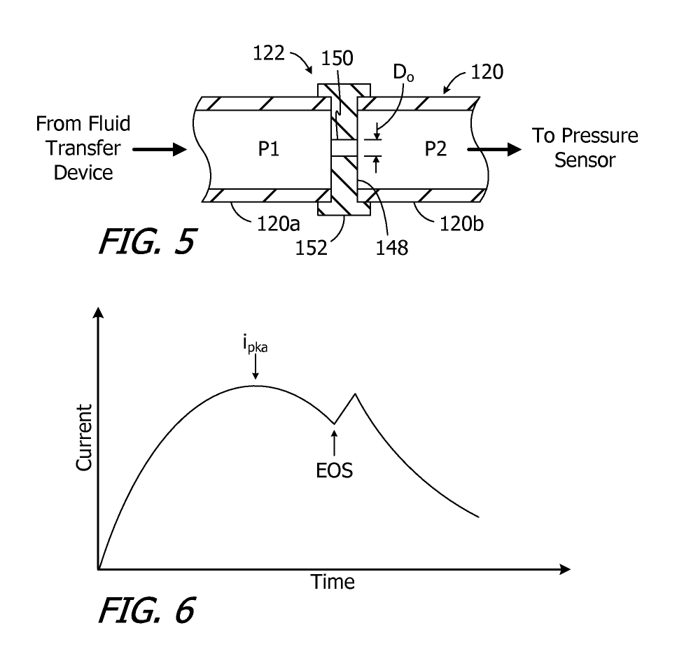

The exemplary ambulatory infusion device 100 illustrated in FIGS. 1-4 also includes a fluid transfer device 114. The inlet of the fluid transfer device 114 is coupled to the interior of the reservoir 110 by a passageway 117, while the outlet of the fluid transfer device is coupled to an outlet port 118 by a passageway 120. A flow restrictor 122 may be positioned along the passageway 120 between the outlet of the fluid transfer device 114 and a pressure sensor 144 (described below). Operation of the fluid transfer device 114 causes infusible substance to move from the reservoir 110 to the outlet port 118. A catheter 124 may be connected to the outlet port 118 so that the infusible substance passing through the outlet port will be delivered to a target body region in spaced relation to the infusion device 100 by way of the outlet 125 at the end of the catheter. As is discussed in greater detail below with reference to FIG. 5-9, the pressure differential across the flow restrictor 122 may be used to measure the volume of fluid flowing from the fluid transfer device 114 and, accordingly, to determine whether the patient is receiving the intended volume of medication or other infusible substance.

A wide variety of fluid transfer devices may be employed. In the illustrated embodiment, the fluid transfer device 114 is in the form of an electromagnet pump that has, among other things, an electromagnet with a core and a coil as well as an armature with a pole and a piston. One example of such an electromagnet pump is illustrated and described in U.S. Patent Pub. No. 2008/0234639, which is incorporated herein by reference. The present inventions are not, however, limited to electromagnet pumps and may include other types of fluid transfer devices. Such devices include, but are not limited to, other electromagnetic pumps, solenoid pumps, piezo pumps, and any other mechanical or electromechanical pulsatile pump. Additionally, in the context of positive pressure reservoirs, the fluid transfer device may be in the form of an accumulator which includes a variable volume housing and active inlet and outlet valves. In the exemplary context of implantable drug delivery devices, and although the volume/stroke magnitude may be increased in certain situations, the fluid transfer devices will typically deliver about 1 microliter/stroke or other actuation, but may be more or less (e.g. about 0.25 microliter/actuation or less) depending on the particular fluid transfer device employed. Additionally, although the exemplary fluid transfer device 114 is provided with internal valves (e.g. a main check valve and a bypass valve), valves may also be provided as separate structural elements that are positioned upstream of and/or downstream from the associated fluid transfer device.

Energy for the fluid transfer device 114, as well for other aspects of the exemplary infusion device 100, is provided by the battery 126 illustrated in FIG. 2. In the specific case of the fluid transfer device 114, the battery 126 is used to charge one or more capacitors 128, and is not directly connected to the fluid transfer device itself. The capacitor(s) 128 are connected to an electromagnet coil in the fluid transfer device 114, and disconnected from the battery 126, when the electromagnet coil is being energized, and are disconnected from the electromagnet coil and connected to the battery when the capacitor(s) are being recharged and/or when the fluid transfer device is at rest. The capacitor(s) 128 are carried on a board 130. A communication device 132, which is connected to an antenna 134, is carried on the same side of the board 130 as the capacitor(s) 128. The exemplary communication device 132 is an RF communication device. Other suitable communication devices include, but are not limited to, oscillating magnetic field communication devices, static magnetic field communication devices, optical communication devices, ultrasound communication devices and direct electrical communication devices.

A controller 136 (FIG. 4), such as a microprocessor, microcontroller or other control circuitry, is carried on the other side of the board 130. The controller controls the operations of the infusion device 100 in accordance with instructions stored in memory 138 and/or provided by an external device by way of the communication device 132. For example, the controller 136 may be used to control the fluid transfer device 114 to supply fluid to the patient in accordance with, for example, a stored delivery profile or a bolus delivery request (generically referred to as a “delivery instruction”). The controller 136 may also be used to monitor and/or calculate pressure, to calculate the volume of fluid supplied with each actuation of the fluid transfer device 114, and to perform the analytical functions described below.

Referring to FIGS. 1, 2 and 4, the exemplary infusion device 100 is also provided with a side port 140 that is connected to the passageway 120 between the outlet of the fluid transfer device 114 and the outlet port 118. The side port 140 facilitates access to an implanted catheter 124, typically by way of a hypodermic needle. For example, the side port 140 allows clinicians to push fluid into the catheter 124 and/or draw fluid from the catheter for purposes such as checking catheter patency, sampling CSF, injecting contrast dye into the patient and/or catheter, removing medication from the catheter prior to dye injection, injecting additional medication into the region at the catheter outlet 125, and/or removing pharmaceuticals or other fluids that are causing an allergic or otherwise undesirable biologic reaction.

The outlet port 118, a portion of the passageway 120, the antenna 134 and the side port 140 are carried by a header assembly 142. The header assembly 142 is a molded, plastic structure that is secured to the housing 102. The housing 102 includes a small aperture through which portions of the passageway 120 are connected to one another, and a small aperture through which the antenna 134 is connected to the board 130.

The exemplary infusion device 100 illustrated in FIGS. 1-4 also includes a pressure sensor 144 that is connected to the passageway 120 between the outlet of the fluid transfer device 114 and the outlet port 118. As such, the pressure sensor 144 senses the pressure at the outlet port 118 which, in the illustrated embodiment, is also the pressure within the catheter 124. The pressure sensor 144 is connected to the controller 136 and may be used to analyze a variety of aspects of the operation of the exemplary implantable infusion device 100, as is described in greater detail below with reference to FIGS. 5-9. An audible alarm 146, which is located within the housing 102, is also connected to the controller 136.

As alluded to above, a flow restrictor may be positioned along the passageway 120, downstream from the fluid transfer device 114 and upstream from the pressure sensor 144. A wide variety of flow restrictors may be employed and, although the present inventions are not limited to any particular type of flow restrictor, one exemplary flow restrictor is illustrated in FIG. 5. The exemplary low restrictor 122, which includes a plate 148, an orifice 150 with a diameter Do, and a mounting flange 152, may be positioned between tubular portions 120a and 120b of the passageway 120. All fluid flowing from the fluid transfer device 114 will pass through the flow restrictor 122. P1 represents the pressure between the fluid transfer device 114 and the flow restrictor 122, while P2 represents the pressure sensed by the pressure sensor 144 downstream from the flow restrictor. Other exemplary flow restrictors include, but are not limited to, capillary tubes and plates, columns packed with particulates that create predetermined pore sizes, filters, and structures with serpentine flow paths.

Volumetric flow of a particular infusible substance through the flow restrictor 122 may be determined, based on the pressure differential across the flow restrictor, with the calculation below. Different calculations may be required for other types of flow restrictors. The calculation is performed by the controller 136 in the illustrated embodiment.

Q=Cf(Ao)\sqrt{2ΔP/ρ}
where
Q=volumetric flow
Cf=flow coefficient of the infusible substance
Ao=area of the orifice

ΔP=P1−P2
ρ=density of the infusible substance.
   As noted above, the pressure P2 downstream from the flow restrictor 122 may be measured directly by the pressure sensor 144. The pressure P1, which is the pressure upstream from the flow restrictor 122, may also be measured by a pressure sensor. In the illustrated embodiment, however, there is no pressure sensor associated with the passageway portion 120a and the pressure P1 is measured indirectly by monitoring the fluid transfer device 114.

In an electromagnet-pump based fluid transfer device, for example, the current flow through the electromagnet coil during each actuation of the electromagnet is indicative of the pressure P1. Accordingly, in the illustrated embodiment, a current sensor 154 (FIG. 4) is connected to the input and output of electromagnet coil and to the controller 136. An exemplary plot of current v. time associated with an actuation of the electromagnet (also referred to as “a current waveform”) is shown in FIG. 6. There are at least four characteristics of the current waveform that may used individually, or collectively with an appropriate algorithm, to determined the pressure P1. The first characteristic is the magnitude of the time period that begins when voltage is applied to the coil and ends when an artifact EOS appears in the current waveform. The artifact EOS indicates that the pump armature has moved from its rest position to its end of stroke position. The second characteristic is the magnitude of the absolute peak current iₚₖₐ. The third characteristic is the magnitude of the time period that begins when voltage is applied to the coil (time=0) and ends when absolute peak current iₚₖₐ occurs. The fourth characteristic is the slope of the curve up to the time at which absolute peak current iₚₖₐ occurs. The average slope of the curve may, for example, be used here. Alternatively, instead of the average slope, the curve may be broken into discrete portions corresponding to short time intervals and the slope of each time interval, or a subset of the time intervals, could be compared to the slopes of the corresponding time intervals on the expected curve. The deviations from the expected slopes may be integrated with respect to time over the portion of the curve up to the time at which the absolute peak current iₚₖₐ occurs (or over the entire curve). The relationship between each or these characteristics and the pressure P1 may be empirically determined, or mathematically modeled, and stored in, for example, the memory 138.

There are a variety of advantages associated with measuring the pressure P1 in this manner. For example, the need for a second pressure to measure the pressure differential across the flow restrictor, which is used to calculate volumetric flow, is eliminated and a savings, both in terms of cost and space, is realized.

The volumetric flow determination may be used by the controller 136 to determine whether the implantable infusion device 100 is supplying the desired volume of fluid to the patient and, if necessary, to dynamically adjust delivery in order to compensate for the factors that are adversely effecting delivery accuracy. For example, compensation values may be derived from the volumetric flow determination and used to increase or decrease the number of future fluid transfer device actuations in order to insure that the intended volume of fluid is delivered to the patient. Such compensation values may be derived periodically, or may be derived after each fluid transfer device actuation. The compensation values, and the manner in which they are applied, will depend upon the type of fluid transfer device, the delivery profile used by the implantable infusion device to dictate the volume of fluid supplied to the patient, and the overall manner in which the fluid transfer device is controlled.

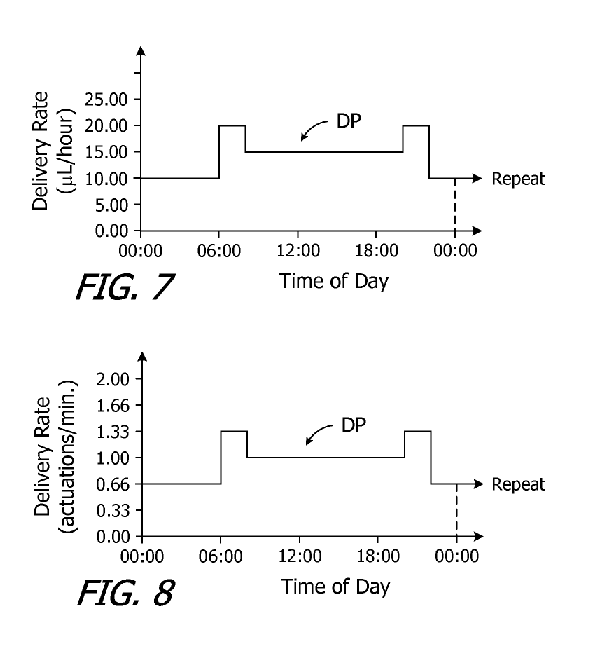

For illustrative purposes only, one exemplary manner of compensating for factors that are adversely effecting delivery accuracy of an implantable infusion device may be described with reference to the exemplary delivery profile that is illustrated in FIGS. 7 and 8. To that end, the exemplary delivery profile DP illustrated in FIG. 7, which has a twenty-four hour cycle time and may be expressed in terms of the volume delivered per hour, may be stored by an implantable infusion device such as the exemplary implantable infusion device 100. The exemplary delivery profile DP specifies that the infusible substance is to be delivered at a rate of 10 microliters/hour (μL/hour) from 00:00 to 06:00 hours, at a rate of 20 μL/hour from 06:00 to 08:00, at a rate of 15 μL/hour from 08:00 to 20:00, at a rate of 20 μL/hour from 20:00 to 22:00, and at a rate of 10 μL/hour from 22:00 to 24:00. In one exemplary infusion device, the fluid transfer device includes an electromagnet pump and each actuation is a pump stroke is expected to result in the delivery of 0.25 μL of infusible substance to the patient. Accordingly, when expressed in terms of actuations per minute in the manner illustrated in FIG. 8, the exemplary delivery profile DP specifies that the infusible substance is to be delivered at a rate of 0.66 actuations/minute from 00:00 to 06:00 hours, at a rate of 1.33 actuations/minute from 06:00 to 08:00, at a rate of 1.0 actuation/minute from 08:00 to 20:00, at a rate of 1.33 actuations/minute from 20:00 to 22:00, and at a rate of 0.66 actuations/minute from 22:00 to 24:00. The conversion from volume per hour to actuations per minute may be performed by the implantable medical device controller 136 and/or by the clinician's programming unit 300 (FIG. 10).

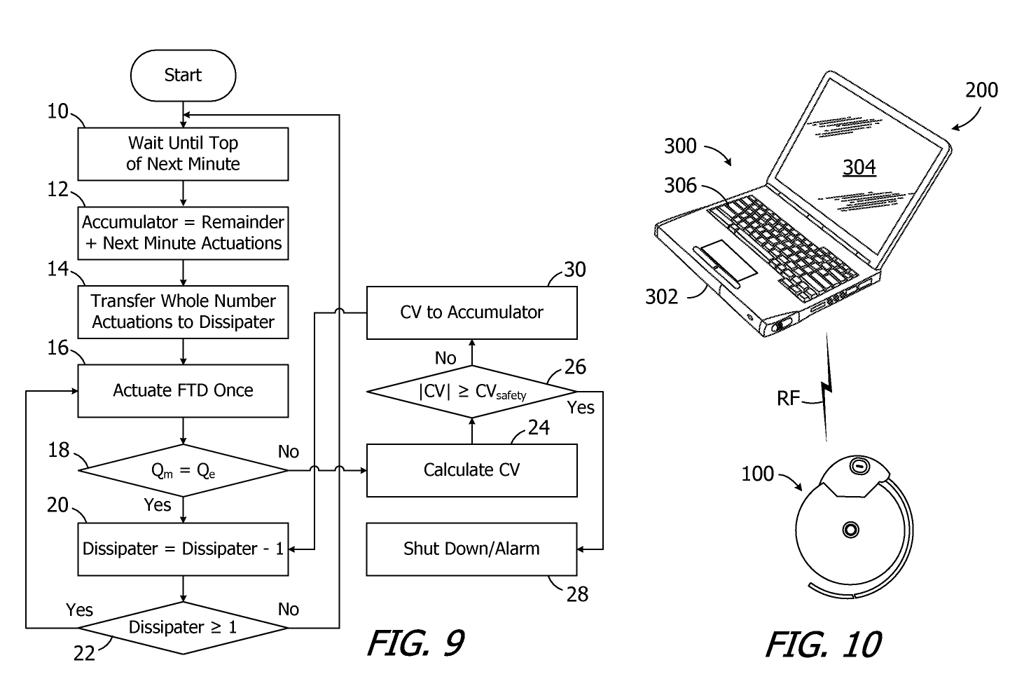

One example of a control method that may be employed by the implantable medical device controller 136 to execute a stored delivery profile, such as the delivery profile DP illustrated in FIGS. 7 and 8, and to also compensate for factors that are adversely effecting delivery accuracy, is illustrated in FIG. 9. The control method actuates the associated fluid transfer device (e.g. fluid transfer device 114), when appropriate, at the top (i.e. beginning) of each minute. If, for example, two pump strokes or other fluid transfer device actuations are required per minute to maintain the requisite flow rate, then those actuations will occur at the top of each minute. It should be noted that the aspect of the exemplary method associated with actuating fluid transfer device in accordance with a delivery profile is merely one example of the manner in which a delivery profile by be implemented and that the present compensation scheme is not limited to this example.

The first step in the exemplary method illustrated in FIG. 9 is to wait until the top of the minute to proceed (Step 10). The second step is to add the actuations associated with this minute (note FIG. 8) to a remainder, if any, in an accumulator (Step 12). If, for example, the rate associated with the minute was 1.33 actuations/minute, then 1.33 actuations would be added to the accumulator. Next, the whole number of actuations in the accumulator is transferred to a dissipater (Step 14). If, for example, 1.33 actuations were in the accumulator, then 1 actuation would transferred to the dissipater and there would be a remainder of 0.33 actuations in the accumulator. The fluid transfer device is then actuated once (Step 16). The volumetric flow Q associated with the actuation is measured (Qₘ) in the manner described above and compared to the expected volumetric flow (Qₑ) (Step 18). If the measured (or “actual”) volumetric flow Qₘ is equal to the expected volumetric flow Qₑ, then it is assumed that the patient is receiving the intended volume and the process will proceed. Specifically, the dissipater will then be decremented by one actuation (Step 20) the actuate/decrement routine will continue so long as there are actuations remaining in the dissipater (Step 22) and Qₘ=Qₑ (Step 18). When there are no actuations remaining in the dissipater, the fluid transfer device will remain in a rest state until at least the top of the next minute (Steps 10 and 22).

If, on the other hand, the measured volumetric flow Qₘ is not equal to the expected volumetric flow Qₑ (Step 18), then the controller 136 calculates a compensation value CV (Step 24). The compensation value CV is a value that represents the difference between the expected volumetric flow Qₑ and the measured volumetric flow Qₘ. In the exemplary context of a fluid transfer device that is expected to supply 0.25 μL per actuation, an actuation that generates 0.0225 μL (90% of the expected per actuation volume) would result in a compensation value CV that represents a deficit of 0.0025 μL (10% of the expected per actuation volume), or one-tenth of one actuation, while an actuation that generates 0.0275 μL (110% of the expected per actuation volume) would result in a compensation value CV that represents a surplus of 0.0025 μL (10% of the expected per actuation volume), or one-tenth of one actuation. The compensation valve CV is used to increase or decrease the future number of actuations of the fluid transfer device, as compared to the number that is associated with the delivery profile, in order to compensate for any deficiencies or surpluses in the measured volumetric flow Qₘ.

Although the present apparatus and methods compensate for such deviations from the expected volumetric flow, it may be assumed for safety purposes that certain compensation values are too large, in absolute terms, to be the result of minor issues and are instead the result of a more serious issue. Serious issues may include, for example, a fluid transfer device failure, a pressure sensor failure, or a particle blocking the flow restrictor orifice. It may also be assumed that is not desirable to allow an implanted medical device to make large increases or decreases in the number of fluid transfer device actuations without input from a physician or other medical professional (collectively “clinician”). By way of example, but not limitation, compensation values that represent more than 15% of the expected volumetric flow may be considered to be too large to be applied to future fluid transfer device actuations absent input from a clinician and may, instead, be the result of a condition that requires immediate attention. Accordingly, the absolute value of the compensation value CV is compared to a predetermined safety value CV_{saftey} in the illustrated implementation (Step 26) and, if |CV|≧CV_{saftey}, then the controller 136 will shut down the fluid transfer device 114 and actuate the alarm 146 (Step 28). It should also be noted here that the alarm 146 may be actuated in other instances. For example, the alarm 146 may be actuated if the pressure measurement taken by the pressure sensor 144 indicates that the catheter 124 is blocked.

In those instances where the compensation value CV is considered to be within an acceptable range (i.e. |CV|<CV_{saftey}), then the compensation value CV may be used to compensate for volumetric flow deficits and surpluses by adding the compensation value CV to the accumulator (Step 30). More specifically, in order to compensate for a volumetric flow deficit (i.e. Qₘ<Qₑ), a positive compensation value, e.g. of one-tenth of one actuation in the example above, would be added to the accumulator. Fluid transfer and volumetric flow monitoring would then continue in accordance with Steps 20, 22, 16 and 18. After ten actuations that result in a positive one-tenth of one actuation compensation value being added to the accumulator, one compensation actuation, in addition to those already called for by the delivery profile, would occur. The additional actuation of the fluid transfer device compensates for the prior deficiencies in volumetric flow and, accordingly, allows the present infusion device to achieve delivery accuracy despite the presence of factors that tend to degrade delivery accuracy. Similarly, in those instances where there is a volumetric flow surplus (i.e. Qₘ>Qₑ), negative compensation values will be added to the accumulator. Ten fluid transfer device actuations in accordance with the example above that result in a compensation value of minus one-tenth of an actuation will, in turn, result in one less actuation than that which is called for by the delivery profile. The reduction in the number of actuations compensates for the prior surpluses in volumetric flow and, accordingly, allows the present infusion device to achieve delivery accuracy. Additionally, whether the inaccuracy is a deficit or a surplus, the present devices and methods will compensate, in what is essentially real time, for transitory factors (e.g. pressure at the outlet port) and ongoing factors (e.g. degradation of the pump or other fluid transfer device) on an ongoing basis.

Implantable infusions devices in accordance with the present inventions, such as the exemplary implantable infusion device 100, may be configured to store data concerning the expected volumetric flow Qₑ, the measured volumetric flow Qₘ, and the compensation value CV associated with all fluid device actuations, or a sampling thereof, in data logs. Such data logs may be used to recalibrate the implantable infusion device 100. The recalibration may be performed by the implantable infusion device 100 itself, or by a clinician who accesses the data logs with a clinician's programming unit in the manner described below.

As noted above, the exemplary implantable infusion device 100 may be included in an infusion device system 200 (FIG. 10) that also includes a clinician's programming unit 300 that is not implanted in the patient. The exemplary clinician's programming unit 300 includes a housing 302, a display 304, a keypad 306, a battery or other power source, a controller, such as a microprocessor, microcontroller or other control circuitry, memory, and a communication device (including an antenna if necessary). Although the present inventions are not limited to any particular communication device, the exemplary communication device is a telemetry device that transmits an RF signal at a specified frequency. The RF signal may, in some instances, be a carrier signal that carries bit streams. The communication device is configured to send signals to and receive signals from the communication device 132 in the implantable infusion device 100 by way of the antenna 134. Other exemplary communication devices include oscillating magnetic field communication devices, static magnetic field communication devices, optical communication devices, ultrasound communication devices and direct electrical communication devices. In some instances, the remote control may also include an audible alarm.

The exemplary clinician's programming unit 300 may be used to perform a variety of conventional control functions including, but not limited to, turning the infusion device ON or OFF and programming various infusion device parameters. Examples of such parameters include, but are not limited to, delivery profile parameters such as the rate of delivery of a given medication, the time at which delivery of a medication is to commence, and the time at which delivery of a medication is to end. Additionally, the implantable infusion device 100 will transmit signals to the clinician's programming unit control 300 to provide status information about the infusion device 100 that may be stored in memory and/or displayed on the display 304. Examples of such status information include, but are not limited to, the state of charge of the battery 126, the amount of medication remaining in the reservoir 110, the amount of medication that has been delivered during a specified time period, and the presence of a catheter blockage. The signals from the infusion device 100 may also be indicative of sensed physiological parameters in those instances where the infusion device is provided with physiological sensors (not shown). Other information includes the aforementioned data logs concerning the expected volumetric flow Qₑ, the measured volumetric flow Qₘ, and the compensation value CV associated with fluid device actuations. The data may be used to, for example, recalibrate the implantable infusion device 100 in general, and the fluid transfer device 114 in particular.

Although the inventions disclosed herein have been described in terms of the preferred embodiments above, numerous modifications and/or additions to the above-described preferred embodiments would be readily apparent to one skilled in the art. By way of example, but not limitation, the present inventions have application in infusion devices that include multiple reservoirs and/or outlets. It is intended that the scope of the present inventions extend to all such modifications and/or additions and that the scope of the present inventions is limited solely by the claims set forth below.

I claim:

1. A method of operating an infusion device which includes a fluid transfer device configured to generate an expected volumetric flow through a fluid flow path when actuated, the method comprising the steps of:
actuating the fluid transfer device in accordance with a delivery instruction that calls for a predetermined number of actuations;
determining the actual volumetric flow associated with at least some of the fluid transfer device actuations; and
adjusting the predetermined number of actuations in response to the actual volumetric flow being different than the expected volumetric flow.

2. A method as claimed in claim 1, wherein the step of determining volumetric flow comprises measuring the pressure differential across a flow restrictor.

3. A method as claimed in claim 2, further comprising the steps of:
directly measuring the pressure at a location in the fluid flow path that is one of upstream from or downstream from the flow restrictor; and
indirectly measuring the pressure at a location in the fluid flow path that is the other of upstream from or downstream from the flow restrictor.

4. A method as claimed in claim 1, wherein the step of determining the actual volumetric flow comprises determining the actual volumetric flow associated with each fluid transfer device actuation.

5. A method as claimed in claim 4, wherein the step of adjusting the predetermined number of actuations comprises calculating a compensation value that represents the difference between the actual volumetric flow and the expected volumetric flow for a single fluid transfer device actuation.

6. A method as claimed in claim 5, further comprising the step of:
adding the compensation value to the predetermined number of actuations.

7. A method as claimed in claim 6, wherein the compensation value comprises a positive compensation value or a negative compensation value.

8. A method of measuring fluid flow in an infusion device with a fluid flow path, the method comprising the steps of:
directly measuring pressure at a location in the fluid flow path that is one of upstream from or downstream from a flow restrictor;
indirectly measuring pressure at a location in the fluid flow path that is the other of upstream from or downstream from the flow restrictor;
determining a pressure differential across the flow restrictor; and
calculating volumetric fluid flow based on the pressure differential.

9. A method as claimed in claim 8, wherein
the step of directly measuring pressure comprises directly measuring pressure in the fluid flow path at a location that is downstream from the flow restrictor; and
the step of indirectly measuring pressure comprises indirectly measuring pressure at a location in the fluid flow path that is upstream from the flow restrictor.

10. A method as claimed in claim 8, wherein
the infusion device includes a fluid transfer device associated with the fluid flow path;
the step of directly measuring pressure comprises directly measuring pressure at a location in the fluid flow path downstream from the flow restrictor; and
the step of indirectly measuring pressure comprises indirectly measuring pressure at a location in the fluid flow path downstream from the fluid transfer device and upstream from the flow restrictor by monitoring at least one aspect of the operation of the fluid transfer device.

11. A method as claimed in claim 10, wherein the at least one aspect of the operation of the fluid transfer device comprises a current waveform associated with fluid transfer device actuation.

12. A method as claimed in claim 11, wherein
the fluid transfer device comprises an electromagnet-based fluid transfer device including an armature that moves from a rest position to a end of stroke position during actuation;
the current waveform includes an artifact that corresponds to the armature reaching the end of stroke position; and
the at least one aspect of the operation of the fluid transfer device comprises the magnitude of a time period that begins when voltage is applied to the electromagnet and ends when the artifact appears in the current waveform.

13. A method as claimed in claim 11, wherein the current waveform defines an absolute peak current and the at least one aspect of the operation of the fluid transfer device comprises the magnitude of the absolute peak current.

14. A method as claimed in claim 11, wherein the current waveform defines an absolute peak current and the at least one aspect of the operation of the fluid transfer device comprises the magnitude of a time period that begins when voltage is applied to the electromagnet and ends when the absolute peak current occurs.

15. A method as claimed in claim 11, wherein the at least one aspect of the operation of the fluid transfer device comprises the slope of a portion of the current waveform.

16. An infusion device, comprising:
a fluid transfer device configured to create an expected volumetric flow of fluid when actuated; and
a controller, operably connected to the fluid transfer device, configured to actuate the fluid transfer device in accordance with a delivery instruction that calls for a predetermined number of actuations, determine the actual volumetric flow associated with at least some of the fluid transfer device actuations, and adjust the predetermined number of actuations in response to the actual volumetric flow being different than the expected volumetric flow.

17. An infusion device as claimed in claim 16, further comprising:
a fluid flow path associated with the fluid transfer device; and
a flow restrictor within the fluid flow path;
wherein the controller determines the actual volumetric flow based on a pressure differential across the flow restrictor.

18. An infusion device as claimed in claim 17, further comprising:
a pressure sensor at a location in the fluid flow path that is downstream from the flow restrictor;
wherein there is no pressure sensor at a location in the flow path that is upstream from the flow restrictor and downstream from the fluid transfer device.

19. An infusion device as claimed in claim 18, wherein the controller is configured to monitor at least one aspect of the operation of the fluid transfer device and to determine the pressure at the location in the flow path that is upstream from the flow restrictor and downstream from the fluid transfer device based on the monitored aspect of the operation of the fluid transfer device.

20. An infusion device as claimed in claim 19, wherein the at least one aspect of the operation of the fluid transfer device comprises a current waveform associated with the actuation of the fluid transfer device.

21. An infusion device as claimed in claim 20, wherein
the fluid transfer device comprises an electromagnet-based fluid transfer device including an armature that moves from a rest position to a end of stroke position during actuation;
the current waveform includes an artifact that corresponds to the armature reaching the end of stroke position; and
the at least one aspect of the operation of the fluid transfer device comprises the magnitude of a time period that begins when voltage is applied to the electromagnet and ends when the artifact appears in the current waveform.

22. An infusion device as claimed in claim 20, wherein the current waveform defines an absolute peak current and the at least one aspect of the operation of the fluid transfer device comprises the magnitude of the absolute peak current.

23. An infusion device as claimed in claim 20, wherein the current waveform defines an absolute peak current and the at least one aspect of the operation of the fluid transfer device comprises the magnitude of a time period that begins when voltage is applied to the electromagnet and ends when the absolute peak current occurs.

24. An infusion device as claimed in claim 20, wherein the at least one aspect of the operation of the fluid transfer device comprises the slope of a portion of the current waveform.

25. An infusion device as claimed in claim 16, wherein the controller is configured to determine the actual volumetric flow associated with each fluid transfer device actuation.

26. An infusion device as claimed in claim 25, wherein the controller is configured to calculate a compensation value that represents the difference between the actual volumetric flow and the expected volumetric flow for each fluid transfer device actuation.

27. An infusion device as claimed in claim 26, wherein the controller is configured to add the compensation value to the predetermined number of actuations.
